(19) United States
(12) Patent Application Publication
(10) Pub. No.: US 2024/0148172 A1
(43) Pub. Date: May 9, 2024

(54) AROMA CONTAINER AND DRINKING DEVICE HAVING AN AROMA CONTAINER

(71) Applicant: air up group GmbH, Munich (DE)

(72) Inventors: Fabian Schlang, Munich (DE); Tim Jäger, Munich (DE)

(73) Assignee: air up group GmbH, Munich (DE)

(21) Appl. No.: 18/279,836

(22) PCT Filed: Mar. 3, 2022
(86) PCT No.: PCT/EP2022/055392

§ 371 Date: Aug. 31, 2023

(30) Foreign Application Priority Data
Mar. 4, 2021 (DE) 10 2021 202 093.3

(51) Int. Cl. A47G 19/22; A61L 9/12

(52) U.S. Cl. CPC A47G 19/2227; A61L 9/12; A47G 2400/04

(57) ABSTRACT

An aroma container for adding an aroma substance to an air flow flowing through the aroma container comprises an upper wall (12), a lower wall (14) as well as at least one side wall (16, 18), which surrounds the aroma chamber, at least one air inlet opening (48) into the aroma chamber as well as at least one air outlet opening (24) from the aroma chamber, wherein an aroma substance, preferably in the form of an aroma concentrate, is present in the aroma chamber, the aroma substance being preferably applied to a carrier substance (42), wherein the aroma container further comprises a means for improved release of the aroma substance to the air flow flowing through the aroma container.

### FIELD OF THE INVENTION

The invention relates to an aroma container and to a drinking device having an aroma container of this kind.

### PRIOR ART

There is an increasing need to ingest drinking liquids which on the one hand have a pleasant flavor but on the other prevent health risks which could be brought about by the ingestion of aroma substances or stabilizing agents dissolved in the drinking liquid. The ingestion of an increased quantity of calories is also to be avoided.

Water which has been given a slightly fruity aroma has therefore become popular in the last few years. However, undesired additives, such as stabilizing substances and a certain amount of sugar, are also contained in this aromatized water. Many users reject such drinks simply on account of this number of calories.

Since the olfactory sensation plays a significant part in gustatory perception in the consumption of food and beverages, drinking systems to date have attempted to influence the smell perceived while drinking. To that end, aroma elements are proposed which can be attached close to the drinking opening on a drinking container so that the aroma element is in the immediate proximity of the nose of the user, who breathes through the nose while drinking and thereby perceives the aroma.

A drinking device for retronasal ingestion of an aroma substance, comprising an aroma container through which air can flow and which supplies aromatized air to a transport channel for drinking liquid, is known from WO 2019/016096 A1. In this way, the aroma substance is ingested retronasally. During drinking, the aroma substance reaches the mouth of the user together with the drinking liquid and subsequently rises retronasally via the pharynx to the olfactory mucosa, where it is detected by the receptors situated there and is perceived by the user. Advantage is taken of the fact that there is a close correlation between the sense of smell and the sense of taste. The user therefore gains the impression that they are tasting the aroma, even though they are in fact only smelling it retronasally.

### DESCRIPTION OF THE INVENTION

The invention is based on the object of proposing an aroma container for adding an aroma substance to an air flow flowing through the aroma container, which container allows for improved enrichment of the air, which has flowed through, with aroma substance.

This object is solved by an aroma container with the features of claim 1 and by a drinking device with the features of claim 14. Preferred embodiments are apparent from the other claims.

The aroma container according to the invention for releasing an aroma substance to an air flow flowing through the aroma container comprises an upper wall, a lower wall as well as at least one side wall, which surround an aroma chamber, at least one air inlet opening into the aroma chamber as well as at least one air outlet opening from the aroma chamber, wherein an aroma substance is present in the aroma chamber, the aroma substance being preferably applied to a carrier substance; further comprising means for improved release of the aroma substance to the air flow flowing through the aroma container.

According to a preferred embodiment of the invention, the aroma chamber comprises a first section for aroma concentrate and a separate, second section, as well as at least one wick extending from the first section into the second section. In this way, aroma concentrate is continually delivered into the second section of the aroma chamber until the air therein is enriched with aroma substance and ideally saturated. The air flow flowing through the aroma container is guided through the second section and is thus enriched with aroma concentrate.

According to an alternative, preferred embodiment of the invention, the aroma chamber comprises a first section for aroma concentrate as well as a separate, second section filled with air and containing a liquid-separating material. In this regard, the air inlet opening opens into the first section and the air outlet opening is located in the second section. When the air flow is guided through the aroma container, it is guided through the aroma concentrate, where the small air bubbles created by a conventional dispersing member are enriched with the aroma concentrate by absorption. Excessive liquid entrainment from the first section into the second section is prevented by means of a suitable liquid separator, e.g. in the form of a nonwoven filter in the connecting portion between the first section and the second section. Thus, with each gulp, the suction pressure generated by the user is used to finely disperse the air flow flowing into the aroma container, enrich it with aroma concentrate and add the air flow enriched with aroma concentrate to the drinking liquid.

According to a further preferred embodiment of the invention, the aroma container further comprises means for heating the aroma container, preferably an electrically operated heating or a heating which can be chemically activated. Tests have shown that heating the aroma chamber in particular to a temperature between 19.5° C. and 55.3° C., depending on the aroma substance used and its boiling point, supports the phase transition of the aroma substance from the liquid to the gaseous phase. The principle of action used is similar to that known from e-cigarettes. The required energy can be supplied by an external energy supply provided on the aroma container or by coupling to a flow element provided in the associated drinking container.

Alternatively, chemically activated heating of the aroma container can be used, as is known from gel-filled heating pads. However, prior to first use, the aroma container has to be activated for this purpose in that it is mechanically actuated, e.g. pressed at a certain location, or by twisting two sections of the aroma container against each other.

According to an alternative, preferred embodiment, the aroma container comprises an electrically operated evaporator, preferably an ultrasonic nebulizing evaporator. This measure also serves to improve the enrichment of the air flow flowing through the aroma container with aroma substance in order to create a more intensive taste experience.

According to a preferred embodiment of the invention, prior to use the aroma substance is present as a dry substance in a first section of the aroma chamber and an activation liquid is present in a separately provided, second section, wherein the aroma container comprises activators to supply the activation liquid to the first section. In other words, prior to use the aroma substance is present e.g. as spray-dried powder that is chemically more stable than a liquid. Only immediately before use, preferably automatically by inserting the aroma container into a drinking container, the activation liquid present in a separately provided, second section is brought into contact with the dry substance, thereby bringing the aroma container into the ready-to-use, activated state. Activation can also take place by pressing or shaking, so that the two components are mixed.

According to an alternative, preferred embodiment of the invention, the aroma container comprises a carrier substance soaked with aroma concentrate as well as a body freely movable in the aroma chamber, which moves in the aroma chamber during movement of the aroma container. During movement of the aroma container, which automatically happens during the drinking process, the freely movable body can thus collide with the carrier substance soaked with aroma concentrate and thereby increase the release of aroma concentrate. Comparable to a solid body colliding with a liquid-soaked foam material, an increased amount of liquid is thus brought to the surface of the carrier substance so that the air flow flowing through the aroma container can be increasingly enriched with the aroma concentrate.

According to an alternative or supplementary embodiment of the invention, an air delivery member can be provided in the aroma container. In the simplest case, this is a propeller-like air delivery member that can be operated electrically or, comparable to a self-winding clockwork, by moving the aroma container.

According to a preferred embodiment of the invention, the aroma substance is a foamed plastic injected into the aroma chamber of the aroma container, to which an aroma concentrate is applied. The advantage of this solution is that the manufacturing of the aroma container is very simple, since the foamed plastic is injected into the aroma chamber and foams up independently therein. The subsequent application of an aroma concentrate has the advantage that it can only move into an easily accessible, open-pore area of the foamed plastic, from where it can also be easily released again.

According to an alternative embodiment of the invention, the aroma carrier substance is an organic material. Examples of suitable organic materials include fungal mycelium or cellulose, cotton, rice, tea leaves, flower petals or starch.

According to an alternative embodiment of the invention, the carrier substance is provided in the form of piece goods, preferably as granulate. The advantage of this carrier material is that it can be filled into the aroma chamber through conventional powder fillers. Moreover, the aroma substance can already be applied to the carrier material before it is filled into the aroma chamber.

According to an alternative embodiment of the invention, the aroma chamber is essentially annular. A carrier substance is present in the essentially annular aroma chamber, which comprises a solid body soaked with aroma concentrate and preferably consisting of a strip-shaped element which has incisions so as to be able to insert the strip-shaped element into at least one partial section of the annular aroma chamber. The advantage associated therewith is simplified manufacturing of the aroma container, but also more uniform compression of the carrier substance in the annular aroma chamber, whereby e.g. with a nonwoven fabric or foam material in the area of the inner wall and in the area of the outer wall essentially the same material properties prevail, so that the properties of the solid body can be optimally adjusted to a good release of the carrier substance.

Alternatively, the carrier substance can already be brought into a suitable preform by hot pressing, so that it can be inserted more easily into an essentially annular aroma chamber, before the carrier material preferably comprising a nonwoven fabric is soaked with aroma concentrate.

A further preferred embodiment of the aroma container according to the invention provides that the flow path to be covered by the air flowing through the aroma container between the air inlet opening and the air outlet opening is optimized with regard to a particularly advantageous release of the aroma substance. This can be achieved by segmenting at least the air space within the aroma chamber to extend the flow path of the air. In this regard, it is to be noted that the flow channel in the aroma chamber has a preferred ratio of wall surface to length of the aroma chamber. As tests have shown, if a length of the flow path is over approximately 8 cm on average, too much of the aroma substance transported in the air flow settles back on the inner wall surface of the aroma chamber, whereas, if a length of the flow path is below 4 cm on average, ideal release of the aroma substance into the air flow is not possible since the distance is too short to ensure sufficient aroma release at ambient temperature.

According to a further preferred embodiment, the essentially annular aroma chamber is configured in such a way that, although the inlet opening for air and the outlet opening for aromatized air are essentially opposite to each other, the flow path in the aroma container is extended—e.g. by a partition wall within the aroma container—in such a manner that the air not only has to flow through an approximately semi-circular flow path between the inlet opening for air and the outlet opening for aromatized air, but preferably has to completely circle the annular aroma chamber once.

A further preferred configuration of the aroma container provides that the aroma chamber is pressurized. By providing a nozzle in the area of the air outlet opening of the aroma chamber, which can preferably be filled with liquid aroma concentrate, the aroma concentrate can thereby be finely atomized in an aerosol-like manner, whereby the air bubbles that enter the mouth of the user during drinking can be enriched with as much aroma-providing substance as possible, which enables an improved taste experience.

It is particularly preferred to attach an adjustable valve to the air outlet opening of the aroma container so that a very controlled release of the aroma substance into the drinking liquid and thus into the mouth of the user is enabled.

According to an alternative, preferred embodiment of the invention, the aroma concentrate is applied to a spherical body consisting of nonwoven fabric, one or more of which are disposed in the aroma chamber through which an air flow flows. Preferably, the spherical aroma bodies are inserted into the aroma chamber. The flowing air flow must pass through one or more of these bodies soaked with aroma concentrate, so that the air flow is well enriched with aroma substance.

According to an alternative embodiment of the invention, the aroma container comprises an optically acting indicator on the aroma container, which detects the time having passed since activation of the aroma container and/or the amount of overflowing air and comprises an optical display. In other words, the indicator serves to give a user an indication of when the supply of aroma substance in the aroma container is running low, so that the aroma container can be replaced in time before a reduced flavor experience occurs. In the simplest case, the indicator is based on the time having passed since activation. Alternatively, an optical display in the form of a color indicator can be provided, which changes its color depending on the volume of overflowing air and indicates to the user when the supply of aroma substance is running low.

A simplified alternative indicator element may be provided in the form of a sticker comprising various sections. If the content in the aroma container is sufficient for five bottle fillings, for example, the indicator designed as a sticker consists of four sections which are removed individually, e.g. scratched off, upon each new filling so that the number of remaining sections correspondingly indicates to the user how often the bottle can still be refilled.

According to a preferred embodiment of the invention, an RFID tag or another passive electronic component for near-field communication is provided on the aroma container. The advantage of RFID tags or comparable electronic components is their now very low unit price and the fact that they do not require a power supply of their own. However, they can communicate with the smartphone of the user, providing the user with information about the activation state, first activation, the aroma contained in the aroma container or the like. The app installed on a user's smartphone can be used at the same time to conveniently reorder aroma containers and inform the user about newly offered flavors.

The drinking device according to the invention comprises an aroma container according to one of the preceding claims and a volume for drinking liquid as well as a transport channel for drinking liquid, which extends to a drinking end.

In this regard, it is preferred that the drinking device further comprises a head part which can be connected to the aroma container such that at least a part of the aroma container is movable from an activated position to a non-activated position, wherein in the activated position the air outlet opening is in flow connection with a transport channel for drinking liquid, and in the non-activated position there is no flow connection between the air outlet opening from the aroma container and the transport channel for drinking liquid, and preferably the air inlet opening is essentially sealed.

According to a preferred embodiment of the drinking device according to the invention, a flow element is arranged in the transport channel for drinking liquid, which can be set into motion when drinking liquid flows through the transport channel and which is coupled to an air delivery member in the aroma container. In this way, for example, a propeller provided in the aroma container can be activated during the drinking process. The flow element in the transport channel for drinking liquid moves faster the more liquid flows through the transport channel for drinking liquid. If the air delivery member in the aroma container is also movable in accordance with the moving speed of the flow element in the transport channel for drinking liquid, the amount of air flowing through the aroma container can thus also be adjusted to the drinking speed.

The aroma container can be designed to be self-activated upon first insertion into or onto an associated drinking device or be activated by the user prior to insertion. For example, the air inlet opening and air outlet opening of the aroma container can be closed by a suitable sealing layer, which is broken open when the container is inserted for the first time into a suitable receptacle of a drinking device, so that air can flow through, but no loss of aroma substance can occur before the first activation.

One option for the user to activate the aroma container actively is to place two parts that can be rotated against each other in an orientation in which the aroma container can be inserted into a corresponding receptacle in the drinking device. In this position, openings in both housing parts which can be rotated against each other are aligned so that the air inlet opening and the air outlet opening are open. However, the activation movement can also be used to bring about activation in the variants described above of an aroma concentrate present in powder form or of two components to be mixed with each other.

Tests with consumers have shown that an improvement in the taste sensation can also be achieved by the aroma container appealing to further senses, for example by being designed in the shape of a fruit or having a color associated with a particular fruit, such as the color of blackberries.

### BRIEF DESCRIPTION OF THE DRAWINGS

The invention is described below by means of several embodiment examples. The figures show the following:

FIG. 1 shows an overall view of an aroma container according to an exemplary geometry;

FIG. 2 shows a plan view of the aroma container according to FIG. 1;

FIG. 3a

and

FIG. 3b schematically show the working steps in the production of a carrier substance suitable for the aroma container according to FIG. 1;

FIG. 4 schematically shows an alternative production method for a carrier substance to be inserted into an annular aroma chamber;

FIG. 5 shows a first embodiment as to the configuration of the aroma chamber of an aroma container according to FIG. 2;

FIG. 6 shows a second embodiment as to the configuration of the aroma chamber of an aroma container according to FIG. 2;

FIG. 7 shows a third embodiment as to the configuration of the aroma chamber of an aroma container according to FIG. 2;

FIG. 8 shows a fourth embodiment as to the configuration of the aroma chamber of an aroma container according to FIG. 2;

FIG. 9 shows a fifth embodiment as to the configuration of the aroma chamber of an aroma container according to FIG. 2;

FIG. 10 shows a sixth embodiment as to the configuration of the aroma chamber of an aroma container according to FIG. 2;

FIG. 11 shows a seventh embodiment as to the configuration of the aroma chamber of an aroma container according to FIG. 2;

FIG. 12 shows an eighth embodiment as to the configuration of the aroma chamber of an aroma container according to FIG. 2;

FIG. 13 shows a side view of a drinking device according to the invention with screwed-on lid;

FIG. 14 shows a side view of a drinking device according to the invention according to FIG. 2 without a lid; and

FIG. 15 shows a sectional view through the head part of a drinking device.

### WAYS OF IMPLEMENTING THE INVENTION

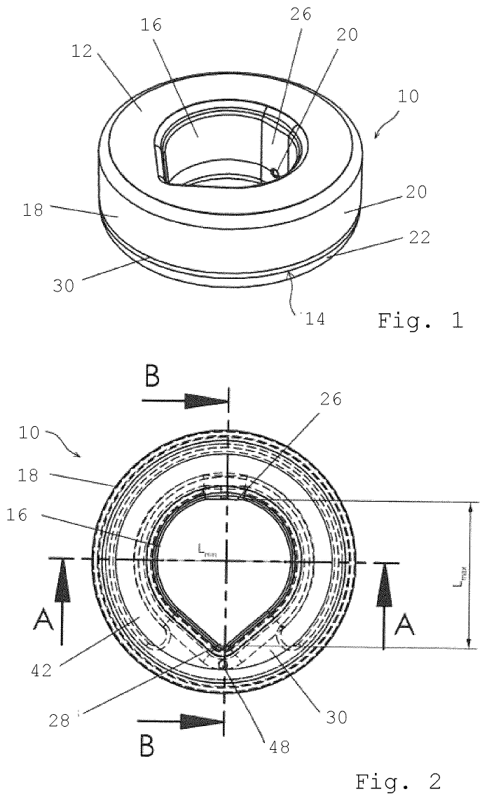

The invention is described below, merely by way of example, by means of the embodiments shown in the figures. In this regard, terms such as upper, lower and lateral/side are used as though the container, for example according to FIG. 1, were to lie with its upper wall on a horizontal, flat surface.

In the respective embodiment examples, a specific geometry of the aroma container shown in FIG. 1 is illustrated. However, the invention is not limited to this geometry. Rather, the invention is applicable to any geometry of aroma containers as long as it has an aroma chamber surrounded by an upper wall, a lower wall and at least one side wall.

The aroma container 10 in this exemplary geometry is shown in more detail in FIGS. 1 and 2. In this regard, the aroma container 10 comprises an upper wall 12, a lower wall 14, an inner side wall 16 and an outer side wall 18. The upper wall 12, lower wall 14, inner side wall 16 and outer side wall 18 surround an aroma chamber 40, as illustrated in FIG. 2.

The aroma container 10 is essentially annular, comprising a circular ring-shaped outer side wall 18 and an inner side wall 16 which deviates from a circular shape. In other words, the space surrounded by the inner side wall 16 has a cross-sectional area which is not circular.

In the specific example, as is most clearly apparent from the illustration according to FIG. 2, the inner side wall 16 is provided with a flat portion 26 and is otherwise approximately drop-shaped, having an essentially tapered end 28. As apparent from FIG. 2, the illustrated geometry of the inner side wall 16 allows for clear positioning of the aroma container 10 on a drinking device (not shown). In this regard, the aroma container 10 can be placed on a correspondingly shaped element of the drinking device, the outer contour of which follows the geometry of the inner side wall 16 to such an extent that the aroma container 10 can be placed on the drinking device only at one single angular position, i.e. with respect to a rotation in the drawing plane of FIG. 2. The flat portion 26 makes it possible to seal the air outlet opening 24 when the aroma container is pushed onto a correspondingly shaped geometry of the drinking device. Providing a flat partial surface 26 in the area of the air outlet opening 24 is more suitable for this than a rounded surface.

The air outlet opening 24 is provided in the seam area between an upper shell 20 and a lower shell 22. This has the advantage that the aroma container can be easily manufactured since no separate slider has to be provided in the case of manufacture by injection molding, but rather the upper shell 20 and lower shell 22 together form the air outlet opening 24.

A shadow gap 30, which fulfills various tasks, is provided in the area between the upper shell 20 and lower shell 22. Firstly, providing a shadow gap 30 between the upper shell 20 and lower shell 22 can prevent that material which escapes during welding of the upper shell 20 to the lower shell 22 impairs the optical appearance of the aroma container. Furthermore, providing the shadow gap 30 makes it possible to achieve rounded edges.

The air inlet opening 48 is schematically shown in FIG. 2 and is located in the lower wall 14 of the aroma container. In this regard, the air inlet opening 48 is preferably located in the lower wall, in the area of the tapering end 28, and thus diametrically opposite to the air outlet opening 24. Consequently, the air flow flowing through the aroma container 10 has to travel as long a travel distance as possible, and can thus be enriched with aroma substance in a particularly effective manner.

As apparent from FIG. 2, a carrier substance 42 carrying the aroma substance is inserted into the aroma chamber 40. In this regard, the carrier substance 42 is U-shaped, i.e. not completely circumferential, and consequently it can be simplified, during the production process, to insert the carrier substance into the lower shell 22 of the aroma container by means of a machine before placing the upper shell 20 thereonto.

The carrier substance 42 can be configured in the form of a foam material or nonwoven fabric.

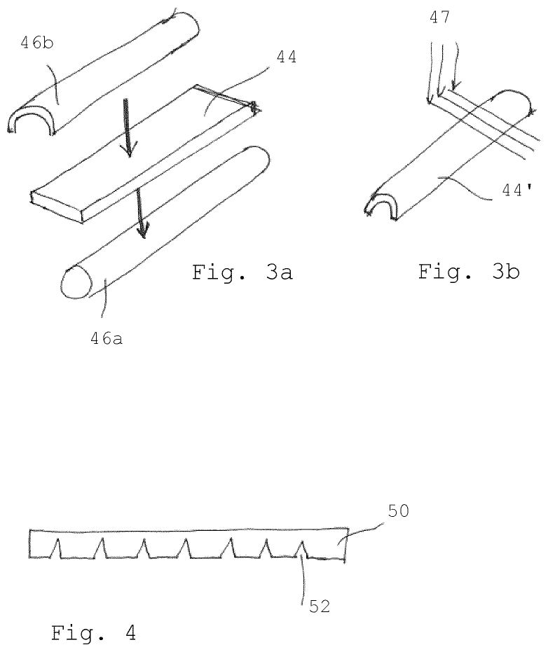

An advantageous option of manufacturing the essentially U-shaped carrier substance 42 is schematically illustrated in FIGS. 3a and 3b. In this regard, an initially flat strip 44 of starting material, e.g. made of foam material or nonwoven fabric, is molded between the elements 46a and 46b of a hot press, so that the geometry of the molded strip 44′ results, as illustrated in FIG. 3b. In a subsequent step, the material is subdivided into individual U-shaped sections along equidistant cutting lines 47, each of which can be inserted into the aroma chamber of an aroma container.

An alternative manufacturing variant according to FIG. 4 is to provide an also strip-shaped starting material 50 provided with incisions 52, the number and geometry of which is adjusted to the desired flexibility of the strip-shaped starting material, so that it can be comfortably inserted into an annular aroma chamber or parts thereof.

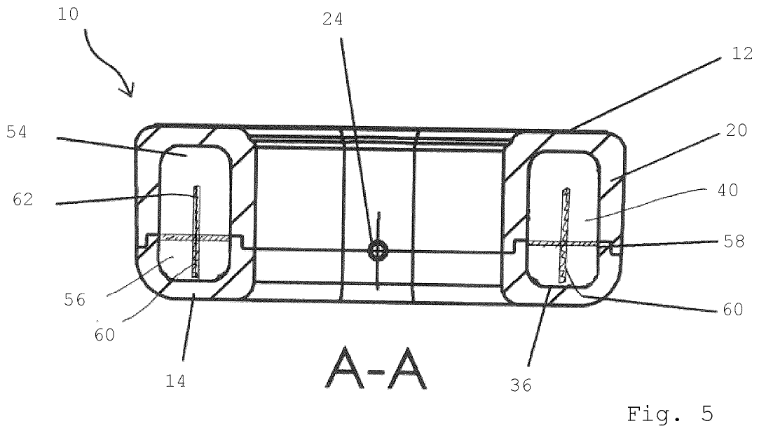

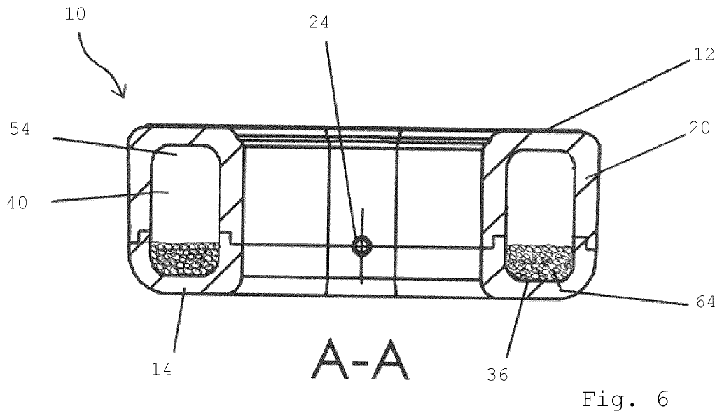

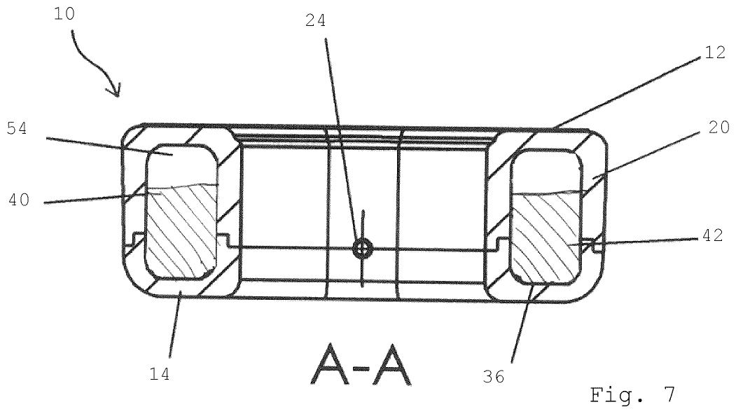

In the embodiment according to FIG. 5, the aroma chamber 40 is divided into an air space 54 as well as a liquid-filled space 56 in which liquid aroma substrate is present. The air space 54 is separated from the liquid-filled space 56 by a boundary wall 58 which in turn has openings into each of which a wick 60 can be inserted. In this regard, the lower end of each wick 60 dips into the aroma substance, preferably an aroma substrate, present in the space 56, whereas the upper end 62 of each wick extends into the air space 54 and, comparable to commercially available room fragrancers, delivers the aroma concentrate into the air space and enriches the air space with the aroma concentrate.

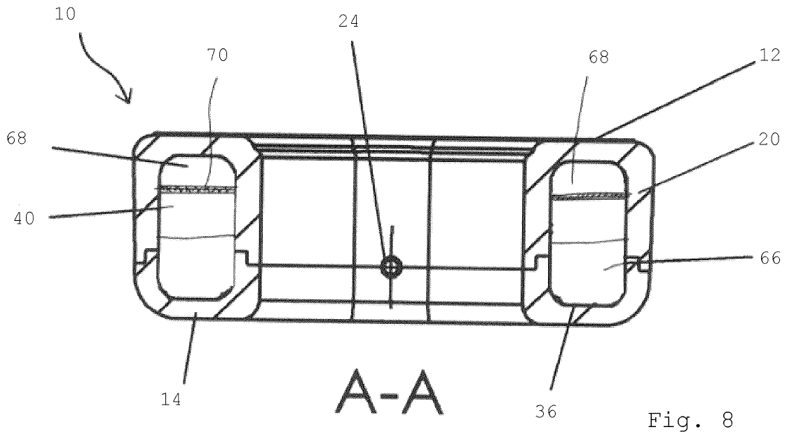

An alternative option, as illustrated in FIG. 6, of bringing about an effective distribution of the aroma substance in the air space 54 is to fill the carrier substance into the aroma chamber in the form of piece goods, preferably in the form of granulate 64, wherein the granulate can already be provided with aroma concentrate prior to filling in said granulate.

An alternative design, instead of filling in piece goods, is to inject foamed plastic into the aroma chamber 40 and apply the aroma concentrate thereto.

As an alternative to foamed plastic, an organic material can also be present in the aroma chamber 40 as a carrier substance 42, whereby a fungal mycelium, cellulose, cotton, rice, tea leaves, flowers, starch or also other suitable organic materials can be used as an organic material, for example. This is illustrated, by way of example, in FIG. 7.

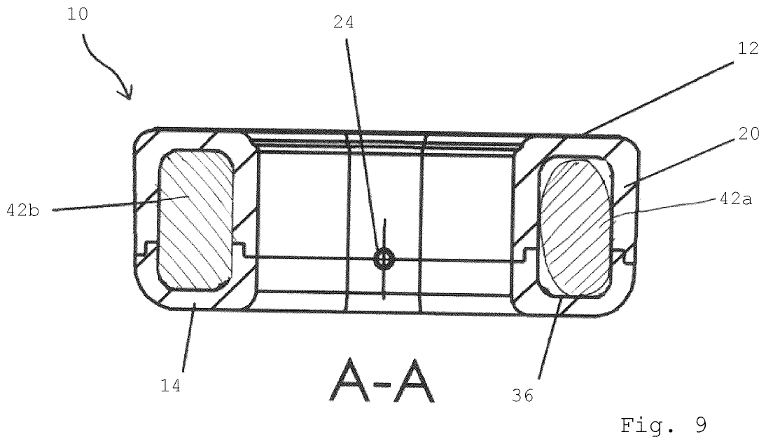

If the aroma substance is unstable in the liquid state, it is advisable to introduce it in dry form, for example as a spray-dried powder, as illustrated in FIG. 8. In this regard, a powder 66, which preferably consists of spray-dried aroma substance, is filled into the aroma chamber 40 in a first section 67. Moreover, another chamber 68 is formed which contains an activation liquid, in the simplest case water, and is separated from the powder 66 by a wall 70. This wall 70 can be opened immediately before use, for example, by pressing or shaking the aroma container 10, so that the activation liquid comes into contact with the powder 66, resulting in a liquid aroma concentrate.

In the embodiment illustrated in FIG. 9, the carrier substance 42 is not provided as an individual element, but in the form of a plurality of individual elements 42a, 42b etc., which only sectionally fill the cross-section of the aroma chamber completely, as in the example of section 42b, or also only essentially fill the cross-section of the aroma chamber, as in the example of section 42a. After entering through the air inlet opening 48 (see FIG. 2), the air must flow through several partial sections of the carrier substance or along several partial sections of the carrier substance, resulting in good mixing as well as enrichment of the air flow with aroma substance.

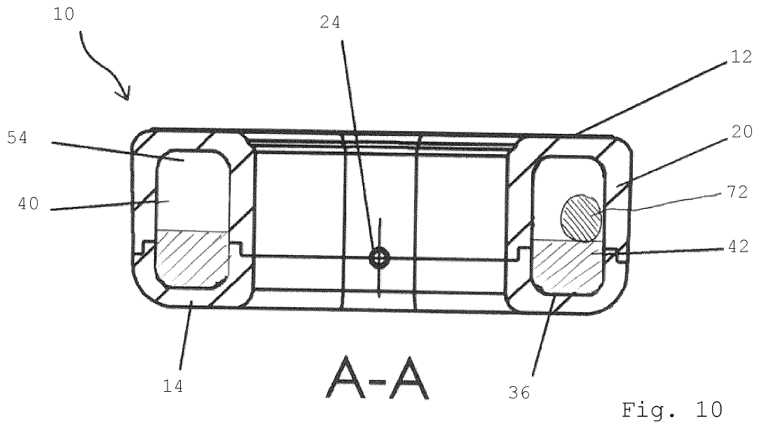

The measure according to FIG. 10 for improving the release of aroma substance consists of providing in the aroma chamber 40, in addition to a carrier substance soaked with aroma concentrate, a plurality of solid bodies 72 which are located in the air space 54 and which, when the aroma container 10 is moved, impact against the walls of the air space as well as against the surface of the carrier substance 42 facing the air space 54 and trigger and enhance the release of aroma concentrate by repeatedly impacting against the carrier substance 42.

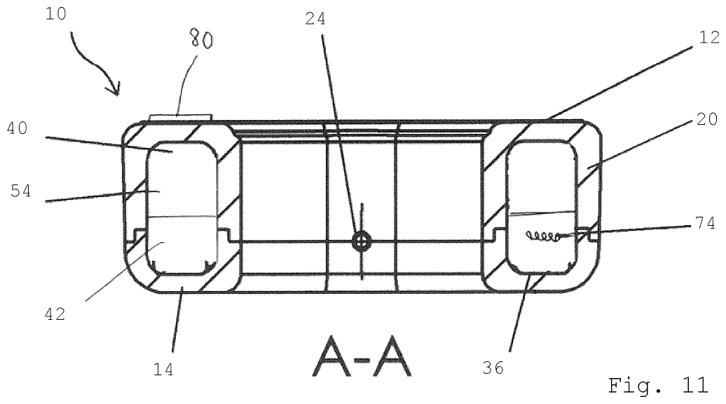

In the embodiment according to FIG. 11, a heating element 74 is provided in the aroma chamber 40, which is supplied with voltage and is located in the area of e.g. a granulate or foamy, nonwoven or organic carrier material and which, by heating in the area of the carrier substance 42, increases the vapor pressure of the aroma concentrate and thus supports the phase transition into the gaseous phase and into the head space 40. In this regard, the most suitable power depends on the number and heat transfer surface of the heating elements as well as the chemical composition of the aroma concentrate, which has a different boiling point depending on the aroma concentrate and therefore requires more or less energy input for local evaporation.

As an alternative to the variant illustrated in FIG. 11 using a current-carrying heating element 74, however, a chemically acting heating element can also be provided, which emits the heat of reaction generated during the exothermic reaction of two substances with each other and thus heats the aroma chamber.

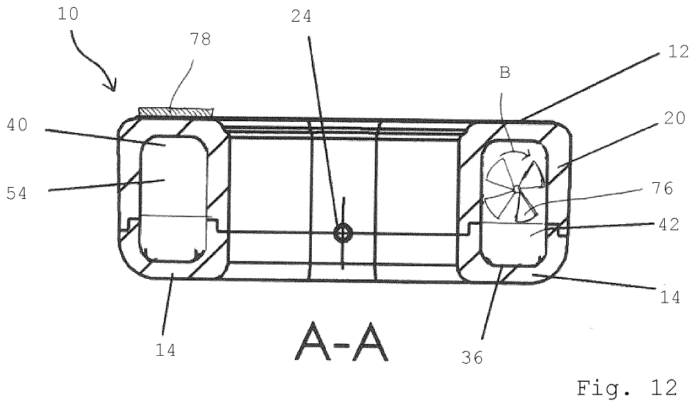

The embodiment illustrated in FIG. 12 can be combined with a large variety of the embodiments discussed above and provides a delivery member 76 in the form of a propeller, as schematically illustrated in FIG. 12, which rotates in the direction of arrow B in the shown example and moves the air flow within the aroma chamber 40 by the inclination of the schematically shown blades. The resulting forced convection enhances mass transfer of the aroma concentrate from the carrier substance 42 into the air space 54.

Completely independent of the illustrated delivery member 76, an indicator 78 is additionally provided in FIG. 12, which is attached to the upper wall 12 of the aroma container 10 and is provided in the form of a sticker having a number of sections to be torn off individually that equals the number of liquid fillings to be aromatized in the drinking bottle associated with the aroma container. After each refill of the bottle, the user can peel off a part of the indicator deck and can thus detect when the aroma substance in the aroma container has been used up so that aromatizing will not be possible the next time to bottle is refilled.

If the aroma container comprises one or more transparent walls, the indicator can also be disposed inside the aroma container. In this case, it is possible to provide an indicator that changes its color when it has been overflowed with a certain volume of air. This indicator can also show the user when the aroma container is depleted or almost depleted.

Apart from a chemical display, the indicator can be activated when the aroma container is inserted for the first time into an associated drinking device and can use an electronic timer to indicate when, also after a period of non-use, the aroma substance is used up and a new aroma container should be inserted into the drinking device.

In FIG. 11, a passive component 80 is moreover provided schematically which does not require a power supply of its own and can transmit information on the operating state and the aroma filling of the aroma container to a user via near-field communication when communicating with a smartphone or smartwatch. Thus, a user cannot only be reminded that the aroma container is almost depleted, but can at the same time be given the option of reordering further aroma containers with the same or different flavors conveniently online. Using a passive component is only illustrated by way of example in connection with FIG. 11 and can be applied in all embodiments of the aroma container according to the invention.

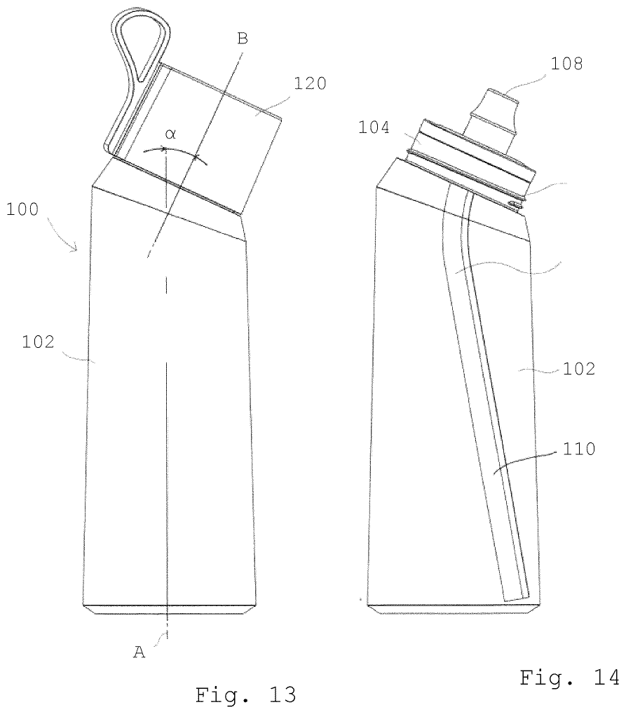

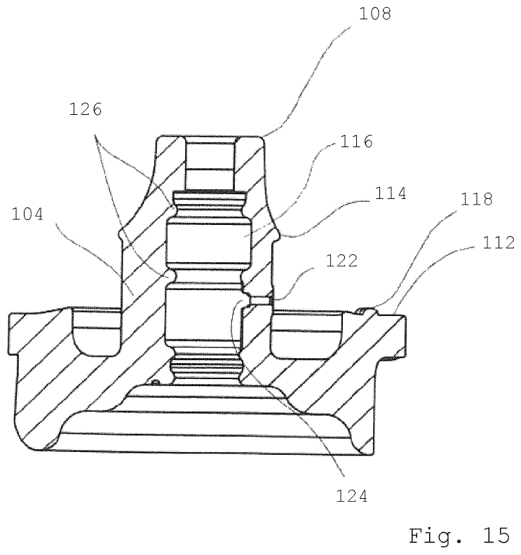

In FIGS. 13 and 14, a drinking device 100 is schematically illustrated in which the essential components are shown by way of example. The drinking device 100 consists of a storage container 102 filled with a pure drinking liquid as well as a head part 104. Herein, a pure drinking liquid is always understood to mean that drinking liquid which does not contain an aroma added by the drinking system according to the invention. The head part 104 comprises a mouthpiece 108 which in this case is integrated in the head part 104 but can also be provided separately. In the head part 104 is an aroma container which is in flow connection with the ambient air in a manner not illustrated in FIG. 14, and from which an air channel for transporting aromatized air leads away. Also provided is a transport channel 110 for drinking liquid, which in the present embodiment example extends like a straw into the pure liquid contained in the storage container 102.

When using the drinking device 100 according to the invention, both the pure liquid and the aromatized air are ingested orally, and the aromatized air proceeds via the retronasal route to the olfactory mucosa, where the aroma is detected by the receptors which are located in the olfactory mucosa, and the user gains the impression, via the neuronal processing of the sensory stimuli, that the pure liquid which the user is drinking has the flavor added by the aroma substance.

In FIG. 11, a section through the head part 104 is illustrated. In this regard, the aroma container is placed over the mouthpiece 108 of the head part 104 and slid onto the head part. The aroma container is supposed to fit tightly against the mouthpiece 188 mainly at the tip and the area diametrically opposite thereto so that there is no play in the area of the openings, but at the same time the friction surface is not too large, in order to facilitate shifting the aroma container in relation to the mouthpiece of the head part 104.

In the inserted state of the aroma container on the mouthpiece 108 of the head part 14, the aroma container is closed and, due to the lower side of the aroma container resting on the area 112 of the head part 104, a non-activated position can be provided depending on the position of the aroma container, in which no air can flow into the air inlet opening of the aroma container. Accordingly, the aroma container can be moved into an activated position which corresponds to the operating position and in which the lower side of the aroma container does not rest on the area 112 and air can flow through the air inlet opening into the aroma chamber.

The stop 114 limits the axial movement of the aroma container when it is pushed up into the operating position. The stop 114 has a flexible structure inwards by a circumferential cavity 116 to enable an easy exchange of the aroma container, for which purpose the aroma container has to be pulled out upwards over the stop 112.

Furthermore, an equalizing channel 118 is provided at the head part, through which air can escape from the storage container and also flow in while drinking. When the lid 120 is in place (see FIG. 13), the equalizing channel 118 is squeezed from above between the edge of the storage container and the lid, thereby providing a seal. When the lid is opened, the equalizing channel 118 vents the excess pressure outwards. At the same time, the equalizing channel 118 is designed in such a way that no water remains in the equalizing channel so that no water can squirt from the opening when the lid of the drinking device is opened.

When it is pushed in, the drinking straw 110 abuts against a stop in the head part. Due to the stop as well as the not circular outer cross-sectional shape of the drinking straw, the position of a hole in the drinking straw is fixed both axially and radially, and is thus positioned in relation to the head part.

An inlet opening 122 is formed in the head part 104. This opening 122 communicates with an equalizing chamber 124 in the head part, which serves as buffer and can equalize overpressure and negative pressure generated during and after drinking and thus prevents water from accidentally flowing into the aroma container. When assembled, the equalizing chamber 124 is formed as a space between the head part 104 and the drinking straw that is not shown in FIG. 15. Suitable stabilization fins are provided within the equalizing chamber 124 to ensure sufficient stability of the head part in the area of the inlet opening 122 so that the desired seal is provided between the aroma container and the head part.

Finally, sealing lips 126 are provided in the mouthpiece of the head part 104, which delimit the different areas in the opening from each other and prevent dirt from entering the functional locations.

1. An aroma container for adding an aroma substance to an air flow flowing through the aroma container, the aroma container comprising:
an upper wall, a lower wall and at least one side wall, which surround an aroma chamber;
at least one air inlet opening into the aroma chamber; and
at least one air outlet opening from the aroma chamber; wherein
an aroma substance is present in the aroma chamber, preferably in the form of an aroma concentrate, the aroma substance being preferably applied to a carrier substance; wherein
the aroma container furthermore comprises a means for improved release of the aroma substance to the air flow flowing through the aroma container.

2. The aroma container according to claim 1, wherein the carrier substance is provided in the form of piece goods, preferably as granulate.

3. The aroma container according to claim 1, wherein the aroma chamber comprises a first section for aroma concentrate as well as a separate, second section, and the means for improved release of the aroma substance to the air flow flowing through the aroma container comprises at least one wick extending from the first section into the second section.

4. The aroma container according to claim 1, wherein the means for improved release of the aroma substance to the air flow flowing through the aroma container comprises means for heating the aroma container, preferably an electrically operated heating or a heating which can be chemically activated.

5. The aroma container according to claim 1, wherein the means for improved release of the aroma substance to the air flow flowing through the aroma container comprises an electrically operated evaporator, preferably an ultrasonic nebulizing evaporator.

6. The aroma container according to claim 1, wherein prior to use the aroma substance is present as a dry substance in a first section of the aroma chamber and an activation liquid is present in a separately provided, second section, wherein the aroma container comprises activators to supply the activation liquid to the first section.

7. The aroma container according to claim 1, further comprising a carrier substance soaked with aroma concentrate, wherein the means for improved release of the aroma substance to the air flow flowing through the aroma container comprises a body freely movable in the aroma chamber, which is movable in the aroma chamber during movement of the aroma container.

8. The aroma container according to claim 1, wherein the carrier substance is a foamed plastic injected into the aroma chamber of the aroma container, to which an aroma concentrate is applied.

9. The aroma container according to claim 1, wherein the carrier substance comprises an organic material, preferably a fungal mycelium, cellulose, cotton, rice, tea leaves or starch.

10. The aroma container according to claim 1, wherein the aroma chamber is essentially annular, and a carrier substance is present in the aroma chamber, which comprises a solid body soaked with aroma concentrate and consisting of a strip-shaped element which has incisions so as to be able to insert the strip-shaped element into at least one partial section of the annular aroma chamber.

11. The aroma container according to claim 1, further comprising an optically acting indicator on the aroma container, which detects the time having passed since activation of the aroma container and/or the amount of overflowing air and comprises an optical display.

12. The aroma container according to claim 1, further comprising an RFID tag or another passive electronic component for near-field communication mounted on the aroma container.

13. The aroma container according to claim 1, wherein the means for improved release of the aroma substance to the air flow flowing through the aroma container comprises an air delivery member in the aroma container.

14. The aroma container according to claim 1, wherein the means for improved release of the aroma substance to the air flow flowing through the aroma container comprises a segmentation within the aroma chamber of at least the air flow, which extends the flow path of the air.

15. A drinking device, comprising:
an aroma container according to claim 1; and
a volume for drinking liquid as well as a transport channel for drinking liquid, which extends to a drinking end; wherein preferably
a flow element is arranged in the transport channel for drinking liquid, which can be set into motion upon flow through the transport channel for drinking liquid and which is coupled to an air delivery member in the aroma container.
